(19) United States
(12) Patent Application Publication
(10) Pub. No.: US 2011/0118446 A1
(43) Pub. Date: May 19, 2011

(54) METHODS AND COMPOSITIONS FOR ANTIBODY PRODUCTION

(75) Inventor: Youngguang Yang, Cambridge, MA (US)

(73) Assignee: The General Hospital Corporation, Boston, MA (US)

(21) Appl. No.: 12/680,253

(22) PCT Filed: Sep. 26, 2008
(86) PCT No.: PCT/US08/77948

§ 371 Date: Sep. 2, 2010

Related U.S. Application Data
(60) Provisional application No. 60/976,326, filed on Sep. 28, 2007.

(51) Int. Cl. C07K 16/00; A61K 39/395; C12N 5/0781

(52) U.S. Cl. 530/388.15; 424/130.1; 435/378; 424/159.1; 424/172.1; 424/175.1; 530/389.1

(57) ABSTRACT

Methods and compositions for antibody production are described herein to produce human antibodies in immunodeficient mice. Particularly mice are implanted with human fetal thymus cells or human thymus and human liver tissue and human hematopoietic cells followed by immunization with an antigen. Immunization involves exposure to any of a variety of antigens including tumor antigens, microbial antigens, viral antigens and/or cytokines and growth factor.

### FEDERALLY SPONSORED RESEARCH OR DEVELOPMENT

The work described below was funded, at least in part, by Grant No. P01 AI 045897 and P30 AI 060354, which were awarded by the National Institutes of Health. The Government may, therefore, have certain rights in the invention.

### TECHNICAL FIELD

This invention relates to antibody production, and production of human antibodies, in particular.

### BACKGROUND

Small animal models allow for systematic in vivo immunological studies. Human thymopoiesis and human T cell development in a small animal model has been achieved by transplantation of human CD34+ cord blood cells into BALB/c RAG2_{nullγCnull} or NOD/SCID/_{γCnull} newborn mice (Traggiai et al., Science, 304:104-107, 2004; Ishikawa et al., Blood, 106:1565-1573, 2005). However, transplantation of hematopoietic stem/progenitor cells has been inefficient in achieving human T cell development in adult mice. Although transplantation of fetal human thymus (Thy) and liver (Liv) tissues can lead to human thymopoiesis and T cell development in NOD/SCID mice, complete hematopoietic repopulation is not achieved in this model (McCune et al., Immunol Rev., 124:45-62, 1991).

### SUMMARY

The invention includes novel methods for producing antibodies. The methods allow reconstitution of a functional human immune system in mice, thereby permitting production of fully human antigen-specific, high affinity antibodies.

In one aspect, the invention features a method for producing human antibodies in a mouse. The method includes implanting human fetal thymus or human fetal thymus and liver tissue in an immunodeficient mouse (e.g., a NOD/SCID mouse, a SCID mouse, or a NOD/SCID/γc knockout mouse), implanting human hematopoietic stem/progenitor cells in the mouse, and immunizing the mouse with a composition including an antigen, thereby producing human antibodies in the mouse.

The human hematopoietic stem/progenitor cells can include CD34+ fetal liver cells, CD34+ bone marrow cells, or CD34+ cord blood cells. In various embodiments, the human fetal stem/progenitor cells implanted in the mouse are enriched for CD34+ cells, prior to implantation.

The human fetal/thymus liver tissue and human hematopoietic stem/progenitor cells can be implanted on the same day, or on different days. For example, the stem/progenitor cells are implanted 1, 2, 3, 4, 5, 7, 14, or more days after implantation of the fetal thymus/liver tissue.

In general, the mouse is immunized after the hematopoietic stem/progenitor cells are implanted, and at a time when human immune cells (e.g., T cells and B cells) can be detected. For example, the immunizing can occur at least 1 week, 2 weeks, 4 weeks, 8 weeks, or 14 weeks

The composition used to immunize the mouse can include an adjuvant (e.g., complete Freund's adjuvant, incomplete Freund's adjuvant, QS21 (from Q. saponaria), a lipopolysaccharide derivative, such as monophosphoryl A, a muramyl dipeptide).

The mouse can be irradiated with whole body irradiation, prior to implantation of the human fetal thymus or thymus and liver tissue.

In some embodiments, the human fetal or thymus tissue is implanted between one and twenty-one days after the mouse is irradiated.

In some embodiments, the human fetal thymus or thymus and liver tissue is implanted under the kidney capsule of the mouse.

The hematopoietic stem/progenitor cells can be implanted by intravenous administration. In some embodiments, at least 1×10² hematopoietic stem/progenitor cells are implanted (e.g., at least 1×10³, 1×10⁴, or 1×10⁵ hematopoietic stem/progenitor cells are implanted).

The mouse can be immunized with a composition more than once (e.g., two, three, or four times).

The method can further include obtaining cells from the mouse, and producing monoclonal antibody-producing cell lines from the cells.

Mice prepared according to the new methods are useful for producing antibodies against a wide variety of antigens. The antigen can be a tumor-associated polypeptide (e.g., one of the following tumor-associated polypeptides: 707 alanine proline (707-AP); alpha (α)-fetoprotein (AFP); adenocarcinoma antigen recognized by T cells 4 (ART-4); B antigen (BAGE); β-catenin/mutated (b-catenin/m); breakpoint cluster region-Abelson (Bcr-abl); CTL-recognized antigen on melanoma (CAMEL); carcinoembryonic antigen peptide-1 (CAP-1); caspase-8 (CASP-8); cell-division cycle 27 mutated (CDC27m); cycline-dependent kinase 4 mutated CDK4/m); carcinoembryonic antigen (CEA); cancer/testis (CT) antigen; cyclophilin B (Cyp-B); differentiation antigen melanoma (DAM-6, also known as MAGE-B2, and DAM-10, also known as MAGE-B1); elongation factor 2 mutated (ELF2M); Ets variant gene 6/acute myeloid leukemia 1 gene ETS (ETV6-AML1); glycoprotein 250 (G250); G antigen (GAGE); N-acetylglucosaminyltransferase V (GnT-V); glycoprotein 100 kD (GnT-V); helicase antigen (HAGE); human epidermal receptor-2/neurological (HER-2/neu); HLA-A*0201-R1701 (HLA-A*0201 having an arginine (R) to isoleucine (I) exchange at residue 170 of the α-helix of the α2-domain in the HLA-A2 gene); human papilloma virus E7 (HPV-E7); human papilloma virus E6 (HPV-E6); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose: (β-D-galactosidase 2-α-Lfucosyltransferase (LDLR/FUT); melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC 1); melanoma ubiquitous mutated 1 (MUM-1), melanoma ubiquitous mutated 2 (MUM-2), melanoma ubiquitous mutated 3 (MUM-3); New York-esophageous 1 (NY ESO-1); protein 15 (P15); protein of 190 KD bcr-abl (p190 minor bcr-abl); promyelocytic leukaemia/retinoic acid receptor α (Pml/RARa); preferentially expressed antigen of melanoma (PRAME); prostate specific antigen (PSA); prostate-specific membrane antigen (PSM); renal antigen (RAGE); renal ubiquitous 1 (RU1), renal ubiquitous 2 (RU2); sarcoma antigen (SAGE); SART-1; SART-3; translocation Ets-family leukemia/acute myeloid leukemia 1 (TEL/AML1); triosephosphate isomerase mutated (TPI/m); tyrosinase related protein 1 (TRP-1 or gp75); tyrosinase related protein 2 (TRP2); TRP-2/intron 2 (TRP-2/INT2); Wilms' tumor gene (WT-1).

In another embodiment, the antigen is a microbial antigen. For example, the antigen is a bacterial antigen (e.g., an antigen expressed by one of the following bacteria: Mycobacterium spp. (e.g., Mycobacterium tuberculosis, Mycobacterium leprae), Streptococcus spp. (e.g., Streptococcus pneumoniae, Streptococcus pyogenes), Staphylococcus spp. (e.g., Staphylococcus aureus), Treponema (e.g., Treponema pallidum), Chlamydia spp., Vibrio spp. (e.g., Vibrio cholerae), Bacillus spp. (e.g., Bacillus subtilis, Bacillus anthracis), Yersinia spp. (e.g., Yersinia pestis), Neisseria spp. (e.g., Neisseria meningitides, Neisseria gonorrhoeae), Legionella spp., Bordetella spp. (e.g., Bordetella pertussis), Shigella spp., Campylobacter spp., Pseudomonas spp. (e.g., Pseudomonas aeruginosa), Brucella spp., Clostridium spp. (e.g., Clostridium tetani, Clostridium botulinum, Clostridium perfringens), Salmonella spp. (e.g., Salmonella typhi), Borrelia spp. (e.g., Borrelia burgdorferi), Rickettsia spp. (e.g., Rickettsia prowazeki), Mycoplasma spp. (e.g., Mycoplasma pneumoniae), Haemophilus spp. (e.g., Haemophilus influenzae), Branhamella spp. (e.g., Branhamella catarrhalis), Corynebacteria spp. (e.g., Corynebacteria diphtheriae), Klebsiella spp. (e.g., Klebsiella pneumoniae), Escherichia spp. (e.g., Escherichia coli), and Listeria spp. (e.g., Listeria monocytogenes).

Alternatively, the microbial antigen is a viral antigen (e.g., a viral antigen encoded by one of the following viruses: human immunodeficiency virus, hepatitis B virus, hepatitis C virus, hepatitis A virus, smallpox, influenza viruses, human papilloma viruses, adenoviruses, rhinoviruses, coronaviruses, herpes simplex virus, respiratory syncytial viruses, rabies, and coxsackie virus).

In various embodiments, the viral antigen is chosen from the group consisting of the following: influenza antigens such as haemagglutinin (HA), nucleoprotein (NP), matrix protein (MP1); HIV antigens such as HIV gag, poi, env, tat, reverse transcriptase hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, and other Japanese encephalitis viral antigen components; rabies viral antigens such as rabies glycoprotein, rabies nucleoprotein and other rabies viral antigen components; and Hepatitis B surface antigen.

In other embodiments, the microbial antigen is a fungal antigen (e.g., an antigen of one of the following fungal species: Candida spp. (e.g., Candida albicans), Cryptococcus spp. (e.g., Cryptococcus neoformans), Aspergillus spp., Histoplasma spp. (e.g., Histoplasma capsulatum), Coccidioides spp. (e.g., Coccidioides immitis), Pneumocystis (e.g., Pneumocystis carinii), Entamoeba spp. (e.g., Entamoeba histolytica), Giardia spp., Leishmania spp., Plasmodium spp., Trypanosoma spp., Toxoplasma spp. (e.g., Toxoplasma gondii), Cryptosporidium spp., Trichuris spp. (e.g., Trichuris trichiura), Trichinella spp. (e.g., Trichinella spiralis), Enterobius spp. (e.g., Enterobius vermicularis), Ascaris spp. (e.g., Ascaris lumbricoides), Ancylostoma spp., Stongyloides spp., Filaria spp., and Schistosoma spp).

In still other embodiments, the antigen is an immunomodulatory molecule (e.g., a cytokine (e.g., TNF-alpha, TGF-beta), a cell surface marker for a lymphocyte (e.g., CD20, CD3, CD2, an integrin, B7, CD23, CD40L, CD19, CD22, CD37).

In some embodiments, the antigen is a growth factor or a growth factor receptor (e.g., vascular endothelial growth factor, epidermal growth factor receptor, HER-2/Erbb2).

The invention also provides human antibodies produced by the methods described herein. The antibodies include polyclonal antibodies, and monoclonal antibodies. Antibodies produced by the methods include high affinity antibodies (e.g., antibodies that bind to the antigen with a K_{D} of 10⁻⁹ M or less) and antibodies of various isotypes and subclasses (e.g., IgM, IgE, IgG such as IgG1, IgG2, IgG3, or IgG4).

As will be appreciated by those of ordinary skill reviewing the present disclosure, many antigens listed above are T-cell dependent antigens in that the development of a strong antibody response to the antigens, when administered as an isolated antigen (rather than, for example, in the context of an organism or tissue) requires T-cell/B-cell interaction. Thus, the present disclosure demonstrates utility of the described model in the preparation of antibodies against T-cell dependent antigens.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Human lymphohematopoietic cell reconstitution in human Thy/Liv/CD34+ FLCgrafted NOD/SCID mice. (A) Levels of total human lymphohematopoietic (CD45+) cells, CD3+ T cells, and CD19+ B cells in PBMCs were analyzed by FCM analysis at 9 weeks after human tissue/cell transplantation. Closed and open bars represent hu-mice that were subsequently used for immunization with DNP₂₃-KLH (n=7) or adjuvant alone (i.e., PBS controls; n=5), respectively. (B,C) Levels of human CD3+ cells (B) and human CD19+ cells (C) in PBMCs, spleen and lymph nodes of DNP₂₃-KLH-immunized (n=7) and PBS-injected control (n=5) hu-mice were analyzed by FCM analysis at time of sacrifice (i.e., week 2 or week 4 postbooster immunization). (D) White pulp formation in hu-mouse spleen. Shown are sections prepared from a representative hu-mouse spleen stained with H&E, anti-human CD3, CD20, and CD68.

FIG. 2. Antigen-specific T cell and antibody responses in immunized hu-mice. (A) Proliferation of human CD3+ T cells in response to KLH (left) and Con A (right). Stimulation index of each individual hu-mouse in DNP₂₃-KLH-immunized (closed circles) and control (open circles) groups are shown. (B) Serum levels of DNP-specific IgG in DNP₂₃-KLH immunized (closed circles) and PBS control (open circles) mice at week 1 after booster immunization (left) and at time of sacrifice (i.e., 2 or 4 weeks after booster immunization; right). Each symbol represents an individual hu-mouse. (C) Serum levels of human IgG1, IgG2, IgG3, and IgG4 in DNP₂₃-KLH immunized (closed circles) and PBS control (open circles) hu-mice at time of sacrifice (i.e., 2 or 4 weeks after booster immunization). Each symbol represents an individual mouse. Like reference symbols in the various drawings indicate like elements.

### DETAILED DESCRIPTION

Co-transplantation of human fetal thymus/liver tissues and CD34+ fetal liver cells into immunodeficient (e.g., NOD/SCID) mice leads to the development of multiple lineages of human lymphohematopoietic cells and formation of secondary lymphoid organs with normal architecture. The invention is based, in part, on the discovery that immunodeficient mice that are “humanized” by implantation of human fetal thymus/liver tissues and CD34+ fetal liver cells can be used to produce antigen-specific antibodies. Humanized mice develop antigen-specific, T cell-dependent antibody responses after in vivo immunization, e.g., with a T-cell dependent antigen. Thus, provided herein are means for producing fully human antibodies in a non-human host.

Animals and human fetal tissues. Immunodeficient mice, such as nonobese diabetic/severe combined immunodeficient (NOD/SCID) mice, are used for implantation of human tissues. Other types of immunodeficient mice may also be used. Human fetal thymus and liver tissues (e.g., of gestational age of 17 to 20 weeks) are obtained for implantation. Mice are conditioned for implantation by treatment with a regimen that deletes bone marrow derived cells, such as irradiation. For example, sublethal (2-3 Gy) whole body irradiation is used. Human fetal thymus and/or liver fragments (e.g., fragments measuring about 0.5-2 mm³) are implanted under the recipient kidney capsule after the treatment to deplete bone marrow derived cells (e.g., within 1-21 days after irradiation, or on the same day as the irradiation). Mice also receive hematopoietic stem/progenitor cells. The hematopoietic stem/progenitor cells administered to the mouse can be CD34+ fetal liver cells (FLCs), CD34+ bone marrow cells, or CD34+ cord blood cells. The hematopoietic stem/progenitor cells can be administered simultaneous with, or after, administration of fetal thymus/liver tissue. Mice can receive (i.v.) hematopoietic stem/progenitor cells (e.g., at least 1,000 cells, e.g., at least 10,000 cells, e.g., 1-5×10⁵/mouse). The hematopoietic stem/progenitor cells can be purified from the same donor as the human thymus/liver tissue.

In some embodiments, the hematopoietic stem/progenitor cells are CD34+ FLCs, which are isolated by the MACS separation system using anti-CD34− microbeads (Miltenyi Biotec, Auburn, Calif.).

Mice can be immunized after human immune cells (e.g., lymphocytes) are detected in the animals. To evaluate chimerism in animals administered the human tissues, levels of human hematopoietic cells can be determined by multicolor flow cytometric (FCM) analysis using various combinations of the following mAbs: anti-HLA class I (W6/32; Leinco Technologies, St. Louis, Mo.), anti-HLA-DR, anti-human CD3, CD4, CD8, CD11c, CD19, CD20, CD45 and CD45RA, anti-mouse CD45, and isotype control mAbs (all purchased from BD PharMingen, San Diego, Calif.). FACS analysis can be performed on a FACScalibur (Becton Dickinson, Mountain View, Calif.).

Antibody production. Monoclonal antibodies (mAbs) can be produced from cells derived from the mice. Monoclonal antibodies may be produced by a variety of techniques, including conventional monoclonal antibody methodology e.g., the standard somatic cell hybridization technique of Kohler and Milstein (1975 Nature, 256:495).

Hybridoma production in the mouse is well established. Immunization protocols and techniques for isolation of immunized splenocytes for fusion are known in the art. Fusion partners (e.g., murine myeloma cells) and fusion procedures are also known.

DNA encoding the heavy and light chain immunoglobulins can be obtained from the murine hybridoma of interest and used to express the immunoglobulins by recombinant means. Mammalian host cells for expressing the recombinant antibodies of the include Chinese Hamster Ovary (CHO cells) (Urlaub and Chasin, 1980 Proc. Natl. Acad. Sci. USA 77:4216-4220; Kaufman and Sharp, 1982 Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

In various embodiments, mice that are “humanized” as described above are immunized twice with antigen in an adjuvant, such as complete Freund's adjuvant or Ribi adjuvant. The antigen/adjuvant composition is administered either in the peritoneal cavity (IP), subcutaneously (Sc) or by footpad (FP). The initial immunizations are followed by additional boosts (e.g., up to 10 immunizations) with the antigen in incomplete Freund's or Ribi adjuvant. The immune response is monitored by retroorbital bleeds. Plasma is screened by ELISA, and mice with sufficient titers of human immunogolobulin against the antigen of interest are used for fusions. Mice are boosted intravenously with antigen 3 and 2 days before sacrifice and removal of the spleen.

Mouse splenocytes, isolated from the immunized mice, are fused with PEG to a mouse myeloma cell line based upon standard protocols. The resulting hybridomas are then screened for the production of antigen-specific antibodies. Single cell suspensions of splenic lymphocytes from immunized mice are fused to nonsecreting mouse myeloma cells with 50% PEG (Sigma). Cells are plated in microtiter plates, followed by incubation in selective medium containing HAT (Sigma). After 1-2 weeks, cells are cultured in medium in which the HAT is replaced with HT. Individual wells are then screened by ELISA for human monoclonal IgG antibodies. Once extensive hybridoma growth occurred, medium is monitored usually after 10-14 days. The antibody secreting hybridomas are replated, screened again and, if still positive for human IgG, antigen-specific monoclonal antibodies are subcloned at least twice by limiting dilution. The stable subclones are then cultured in vitro to generate small amounts of antibody in tissue culture medium for further characterization.

### Examples

Preparation of Hu-Mice and Immunization. Nonobese Diabetic/Severe Combined immunodeficient (NOD/SCID) mice (Jackson Laboratories; Bar Harbor, Me.) were given 2Gy whole body irradiation, transplanted with human fetal Thy/Liv tissues (Advanced Bioscience Resource; Alameda, Calif.) under the kidney capsule, and injected intravenously with CD34+ fetal liver cells (FLCs) isolated from the same fetal donor. Fourteen weeks after transplantation, mice were immunized with 2.4-dinitrophenyl-conjugated keyholelimpethemocyanin (DNP₂₃-KLH, 100 μg/mouse) or an equal volume of PBS (control mice) in complete Freund's adjuvant subcutaneously in dorsal skin, followed by a booster shot (50 μg/mouse of DNP₂₃-KLH or PBS in incomplete Freund's adjuvant) 3 weeks later. Mice were sacrificed for analyses at 2 or 4 weeks after the booster injection.

Analysis of human immune reconstitution, and measurement of human T cell responses and antibody production. Human lymphohematopoietic cell repopulation was analyzed by flow cytometry and immunohistochemistry. Fluorescence-labeled mAbs used for flow cytometric analysis (anti-human CD3, anti-human CD45, anti-human CD19) were purchased from BD Bioscience (San Jose, Calif.), and mAbs used for immunohistochemical staining (anti-human CD3, anti-human CD20, anti-human CD68) were purchased from DAKO, (Carpinteria, Calif.). To quantify human T cell responses, splenocytes from immunized and control hu-mice were stained with carboxyfluorescein diacetate succinimidyl estersd (CFSE, Invitrogen, Carlsbad, Calif.). Cells were then cultured with medium alone, 12.5 ug/ml KLH, or 2 ug/ml ConA. On the fifth day, cells were stained with APC-conjugated anti-human CD3 mAb (BD Bioscience), and proliferation of human CD3+ cells was analyzed by flow cytometry. Stimulation index (S.I.) of human CD3+ cells is calculated by the following formula: (# of cell divisions x % of cells divided) of stimulated culture/(# of cell divisions x % of cells divided) of medium control culture. The levels of DNP-specific human IgG and its subclass antibodies were measured by ELISA using DNP₂₇-BSA-coated plates and detected by HRP-conjugated antihuman IgG (BETHYL, Montgomery, Tex.), IgG1, IgG2, IgG3, and IgG4 mAbs (AbD serotec, Oxford, UK).

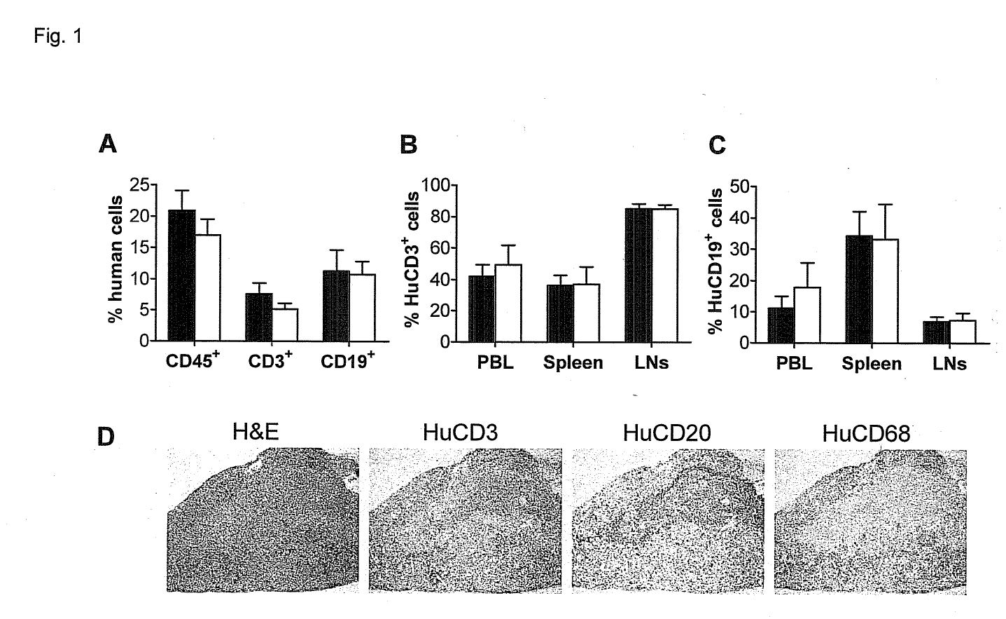

Successful human lymphohematopoietic cell reconstitution was observed in NOD/SCID mice after fetal human Thy/Liv/CD34+ FLC transplantation. Flow cytometric analysis of PBMCs revealed the development of multilineage human lymphohematopoietic cells in the hu-mice prior to immunization (FIG. 1A). Two groups of humice with comparable levels of human cell repopulation were then immunized with T cell dependent antigen DNP₂₃-KLH or PBS. Human chimerism was also analyzed in various tissues, including peripheral blood, spleen, and lymph nodes (LNs) when these hu-mice were sacrificed for measuring T cell responses. As shown in FIG. 1B-C, the immunized and control hu-mice had similar levels of human T cells (FIG. 1B) and B cells (FIG. 1C) in all tissues examined.

Histological analysis confirmed the formation of secondary lymphoid tissues with normal structural feature in the hu-mice. Spleens from these hu-mice were separated into red pulp containing human CD68+ macrophages, and white pulp, in which human T cell areas and B cell follicles were clearly detected (FIG. 1D). Studies have shown that follicular dendritic cells (FDCs) of non-hematopoietic origin play an essential role in the organization of follicular structure. Furthermore, mouse FDCs have been reported to provide costimulation to human lymphoid cells. Thus, it is likely that the cross-species interaction between mouse FDCs and human B and T cells occurred in these hu-mice. However, in these hu-mice most of the white pulp area was occupied by large B cell follicles, which is more similar to the structure of splenic white pulp of primates rather than that of rodents, suggesting that the species-specific white pulp structure is determined largely by bone marrow-derived lymphoid cells, rather than FDCs.

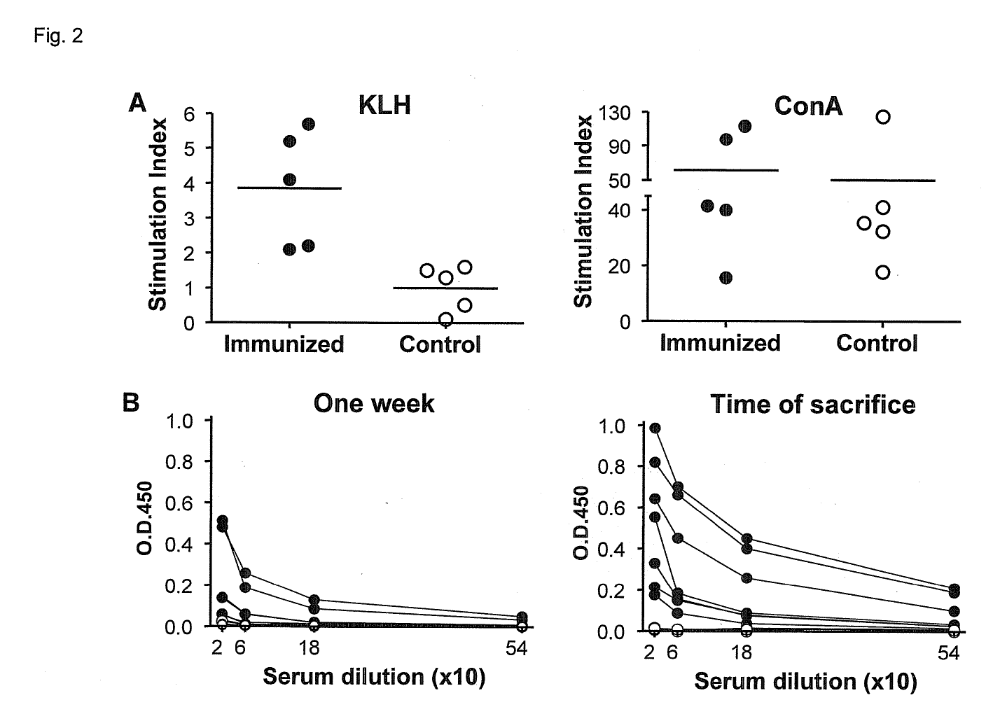

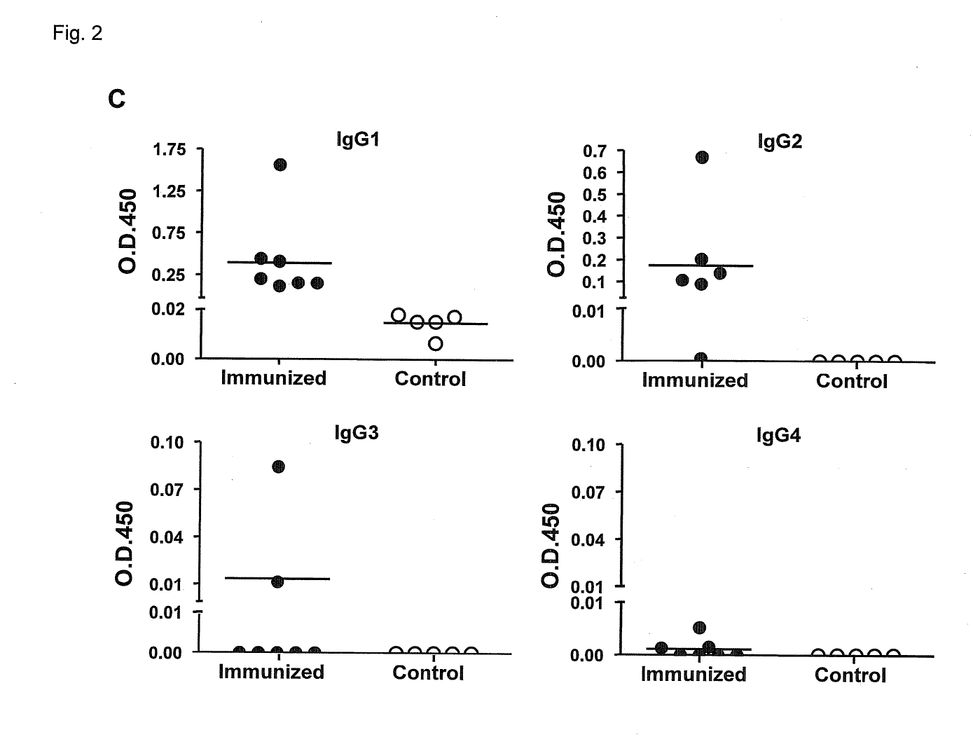

KLH-specific T cell proliferation was analyzed by flow cytometric analysis of CFSE dilution at 2 and 4 weeks after booster immunization. As shown in FIG. 2A, human CD3+ T cells from spleen of DNP₂₃-KLH-immunized, but not from control hu-mice, proliferated in response to KLH stimulation. However, human CD3+ T cells from DNP₂₃-KLH-immunized and control hu-mice showed similar proliferation to Con A stimulation. These results demonstrate that KLH-specific human T cells were primed in vivo in the DNP₂₃-KLH immunized hu-mice.

The levels of DNP-specific human IgG and IgG subclasses in the sera of immunized and control hu-mice were determined by ELISA. As shown in FIG. 2B, 4 of 7 immunized mice showed detectable levels of DNP-specific IgG at week 1 after DNP₂₃-KLH immunization. The levels of DNP-specific IgG in the immunized hu-mice increased over time, and all these mice became positive by the time they were sacrificed at week 2 or 4 post-immunization. In contrast, DNP-specific IgG was not detected in any of PBS control hu-mice. DNP-specific IgG in immunized hu-mice were mainly of the IgG1 and IgG2 subclasses (FIG. 2C). Two hu-mice also showed detectable levels of DNP-specific IgG3, but none was positive for DNP-specific IgG4. In fact, the development and distribution of DPN-specific human Ig subclasses in these immunized hu-mice were similar to that of antibody responses in humans after KLH immunization, in which IgG3 antibody production is less frequently and IgG4 antibodies develop very slowly.

These data show clearly that class switch of antigen specific antibodies taken place in these hu-mice. These results also indicate that functional human T-B cell interactions, such as T cell help for B cell activation, antibody production and class switch occurred in these hu-mice. Taken together, the data demonstrate that hu-mice created by cotransplantation of human fetal Thy/Liv and CD34+FLCs can mount strong antigen-specific T cell responses and T cell dependent IgG production after in vivo antigen immunization.

A number of embodiments of the invention have been described. Nevertheless, it will be understood that various modifications may be made without departing from the spirit and scope of the invention. Accordingly, other embodiments are within the scope of the following claims.

1. A method for producing human antibodies in a mouse, the method comprising:
providing an immunodeficient mouse that has been implanted with:
(i) human fetal thymus or human thymus and liver tissue; and
(ii) human hematopoietic stem/progenitor cells in the mouse, and
immunizing the mouse with a composition comprising an antigen, thereby producing human antibodies in the mouse.

2. The method of claim 1, wherein the mouse is a NOD/SCID mouse, a SCID mouse, or a NOD/SCID/γc KO mouse.

3. The method of claim 1, wherein the human hematopoietic stem/progenitor cells comprise CD34+ fetal liver cells, CD34+ bone marrow cells, or CD34+ cord blood cells.

4. The method of claim 1, wherein the human fetal thymus or thymus and liver tissue and human hematopoietic stem/progenitor cells are implanted on the same day.

5. The method of claim 1, wherein the immunizing is performed when human immune cells are detected, after the hematopoietic stem/progenitor cells are implanted.

6. The method of claim 1, wherein the immunizing is at least 8 weeks after the hematopoietic stem/progenitor cells are implanted.

7. The method of claim 1, wherein the immunizing is at least 14 weeks after the hematopoietic stem/progenitor cells are implanted.

8. The method of claim 1, wherein the composition comprises an adjuvant.

9. The method of claim 8, wherein the adjuvant is complete Freund's adjuvant.

10. The method of claim 8, wherein the adjuvant is incomplete Freund's adjuvant.

11. The method of claim 1, wherein the mouse is irradiated with whole body irradiation.

12. The method of claim 1, wherein the human fetal thymus or thymus and liver tissue is implanted between one and twenty-one days after the mouse is irradiated.

13. The method of claim 1, wherein the human fetal thymus or thymus and liver tissue is implanted under the kidney capsule of the mouse.

14. The method of claim 1, wherein the hematopoietic stem/progenitor cells are implanted by intravenous administration.

15. The method of claim 1, wherein at least 1×10² hematopoietic stem/progenitor cells are implanted.

16. (canceled)

17. (canceled)

18. The method of claim 1, wherein the mouse is immunized with the composition more than once.

19. The method of claim 1, further comprising obtaining B cells from the mouse, and producing monoclonal B cell lines from the B cells.

20. The method of claim 1, wherein the antigen is a tumor-associated polypeptide, a microbial antigen, a bacterial antigen, a viral antigen, a fungal antigen, an immunomodulatory molecule, a cell surface marker for a lymphocyte, a growth factor or a growth factor receptor, or a T-cell dependent antigen.

21. (canceled)

22. (canceled)

23. (canceled)

24. (canceled)

25. (canceled)

26. (canceled)

27. (canceled)

28. A human antibody produced by the method of claim 1.

29. A human monoclonal antibody produced by the method of claim 1.
